(19) United States
(12) Patent Application Publication
(10) Pub. No.: US 2021/0378921 A1
(43) Pub. Date: Dec. 9, 2021

(54) ENCAPSULATED REFILL FRAGRANCE DEVICE AND PREPARATION METHOD

(71) Applicant: Jose ABEL VELAZQUEZ MOSQUEDA, Zapopan (MX)

(72) Inventor: Jose ABEL VELAZQUEZ MOSQUEDA, Zapopan (MX)

(21) Appl. No.: 16/328,621

(22) PCT Filed: Sep. 27, 2018
(86) PCT No.: PCT/MX2018/000100

§ 371 Date: Feb. 26, 2019

(51) Int. Cl. A61K 8/11; A61K 8/25; B65D 65/46; A61L 9/04; A61L 9/12

(52) U.S. Cl. CPC A61K 8/11; A61K 8/25; A61L 2209/133; A61L 9/044; A61L 9/12; B65D 65/466

(57) ABSTRACT

The invention described presents a device and the method for obtaining the constituent structures to generate a fragrance supply assembly by means of the absorption of the fragrance in a polymer which, when exposed to an environment, disseminates the fragrance over time within the surrounding environment where said output is gradual. Additionally, the constituent materials of the device are biodegradable.

The specification describes the device and the method for preparing the components and the assembly which allow to aromatize for longer periods of time than the devices that are currently on the market, and without the need to use electrical elements for the release of the fragrance in the desired space with a greater amount of aroma and controlled dosage.

### FIELD OF THE INVENTION

The present invention relates to a device and the method for obtaining the constituent parts to generate the fragrance delivery device by absorbing the fragrance in a polymer, said device, when exposed in a setting, disseminating the fragrance with the passage of the time within a surrounding environment where said output of fragrance is gradual, the constituent materials of the device being additionally biodegradable.

### BACKGROUND OF THE INVENTION

For many years, devices and methods have been used for the supply of fragrance to the environment which seek to deodorize rooms, carpets, etc., to make them more fragrant and pleasant. The previous systems include candles, hot oils, atomizers, diffusers, as well as other mechanisms of fragrance transport, as is the case of nebulization or atomization devices; as an example of the foregoing and as a result of the analysis of the state of the art, document U.S. Pat. No. 8,727,234B2, document U.S. Pat. No. 7,070,121, document U.S. Pat. No. 6,793,149 and 25 document U.S. Pat. No. 8,821,802B2 were found. In addition to the above, tablets and granules impregnated with fragrance have been developed for their insertion in bags for vacuum cleaners, said fragrance being released throughout the rooms from the tablets or granules once the apparatuses are turned on.

On the other hand, there are several compositions in the state of the art which include fragrance materials which, according to an analysis of the state of the art, may be mentioned, but not in a limiting manner, such as U.S. Pat. Nos. 5,160,494 and 5,449,512.

Current methods present several disadvantages, among which is the disadvantage of having an inaccurate dosage, as well as the possibility of contamination of the surfaces of the tank or device with liquid samples and, on the other hand, the complexity of the dosing device.

Recently, it has been proposed that the fragrance cartridges be able to store the fragrance for prolonged periods before releasing it in a controlled manner and that this be time-dependent, which is sometimes takes place by means of a carrier gas. There are examples of fragrance-carrying cartridges such as the one mentioned in document FR2771930B1, in which the ability to release a fragrance in a carrier gas in a controlled time is described. The fragrance is confined to a cylindrical type cartridge of uniform cross section and is supported on a suitable carrier material. The closure of the fragrance carrier cartridge to prevent the loss thereof during storage is made of a flexible membrane disc sealing both ends of the cartridge. The fragrance is released when the membranes are pierced with hollow needles or cannulas, whereby the fragrance flows in a stream of the carrier gas.

With reference to the above, there is a need to provide means of storage and release of fragrances in demand and in a controlled manner over time; even when they can be inactive with mass preparation methods and at a low cost.

A study of fragrance diffusion behavior revealed that the displacement per unit of time of even very diffuse fragrance materials was remarkably small. As a practical guide, it was further found that the cartridges must be configured in such a way that the fragrance drip does not exceed a speed of 400 ng per second.

On the other hand, there are devices that supply fragrance by means of electric heaters and with power supply. By applying heat to the fragrance or perfume source, there will be a constant supply of perfume or fragrance in the space in which the appliance is being placed; this requires energy in the device and a greater complexity of the same.

As mentioned, there is on the market a great diversity of fragrances, which have the peculiarity of having a lifetime of no more than 30 days and which are aimed at different environments such as home, business, and any other type of space or establishment.

As a result of all of the above, the invention which is the object of this document describes a device and a method for preparing the components of the device which allows the release of fragrance for longer times compared with devices currently on the market and without the need to use energy elements for the release of the fragrance in the desired space, with a greater amount of aroma and a controlled dosage.

### BRIEF DESCRIPTION OF THE FIGURES

FIG. 1 shows the component called outer structure of the encapsulated refill fragrance device, in top perspective view.

FIG. 2 shows an exploded view of the components of the refill fragrance device in perspective, where all the component structures of the device can be seen.

FIG. 3 shows a front section of the complete device which comprises the inner structure, the intermediate structure and the outer structure of the encapsulated refill fragrance device.

### DETAILED DESCRIPTION OF THE INVENTION

The characteristic details of the encapsulated refill fragrance device and its method of preparation are clearly shown in the following specification and in the illustrative figures which are annexed, the same reference numbers serving to indicate the same parts. The main feature of the invention is that a polymeric matrix absorbs the fragrance in its three structures, which are assembled and integrated into a single piece that, once set to be used, will later be exposed in an environment; with the passage of time the fragrance 25 will begin to volatilize to be present in the exposed environment, providing a scent with the resulting gradual release of the fragrance.

In order to obtain a long-lasting air freshener, the invention considers the combination of materials and specific technology to achieve the proposed objective.

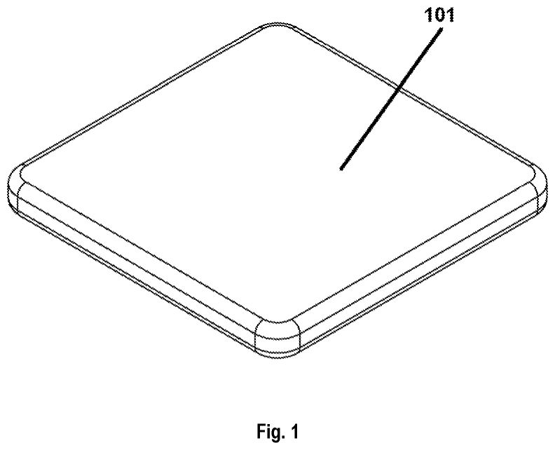

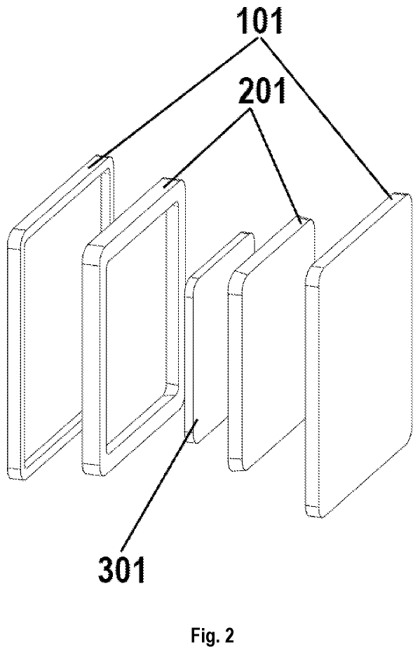

The device is made up of three main structures, with different functional characteristics. The components have different denominations and FIG. 1 shows the component called outer 35 structure (101). FIG. 2, which shows the components of the refill fragrance device, shows the component called inner structure (301) and the component called intermediate structure (201), which form an assembly with the outer structure (101).

The outer structure (101) can be manufactured in various commercial designs, made of plastic, with a color and a fragrance; the intermediate structure (201) as well as the outer structure (101) are made of plastic, with a color and a fragrance; and the inner structure (301), which is obtained by the combination of lignocellulosic fibers, polymer matrices, fragrances, pigment and silicon dioxide, as described in the following paragraphs.

Lignocellulosic fibers are mainly composed of cellulose, hemicelluloses, and lignin, as well as extractables and inorganic compounds. The cellulose fibers are formed by arrangements of cellulose microfibrils; cellulose chains joined by means of hydrogen bridges, in which crystalline sections, called micelles, are intermixed with amorphous regions, joined by an amorphous matrix of lignin. It is believed that the hemicelluloses present in natural fibers fulfill the function of making cellulose and lignin compatible. These fibers, which form part of the invention, are previously treated for the elimination of impurities. They are subjected to processes of defibering, washing, spinning, drying, grinding, sieving and finally dehumidifying. Being a hygroscopic material, the presence of water is important to consider. Therefore, it is dehumidified before being used (70° C. for 3 hours, 60° C. for 5 hours, 50° C. for 7 hours, 40° C. for 10 hours), having less than 5% ranges by mass of water present in the fiber.

Polymers are macromolecules formed by the union of smaller molecules called monomers. To classify polymers, one of the methods used is to heat them above a certain temperature. Depending on whether the material melts and flows or, on the contrary, does not, they are differentiated into thermoplastics or thermosets (thermo-fixes) respectively.

Thermoplastics flow (convert to a liquid state) when heated and re-harden (return to the solid state) when cooled. Their molecular structure presents few (or no) cross-links.

Another classification can be made by considering their elastic behavior. Polymers with an elastic behavior, which can be easily deformed without breaking their bonds or modifying their structure, are called elastomers. They are materials with a very low modulus of elasticity and high extensibility, that is, they deform to a great extent when subjected to pressure, but recover their initial shape by eliminating the pressure. In each cycle of extension and contraction, elastomers absorb energy, a property called resilience.

The fragrance is generated by a complex mixture of gases, vapors and dust, where the composition of the mixture influences the type of smell perceived by the receiver. What we cannot perceive by smell is called odorless. The term fragrance or scent is used mainly by the industry to describe a pleasant smell. The smells correspond to the objective phenomenon of the elements dissolved in the air, although, as in other senses, several psychological factors can play a certain role in their perception.

Fragrances are a mixture of odoriferous substances of natural origin (essential oils) or synthetic (organic products). The essential oils of natural origin in turn can come from the animal kingdom or the plant kingdom. The essences of synthetic origin tend to be organic products such as hydrocarbons, alcohols, aldehydes, ketones, aliphatic and aromatic esters.

Silicon oxide (IV) or silicon dioxide (SiO₂) is a compound of silicon and oxygen, commonly called silica, and is an amorphous substance that is synthetically produced by a process of hydrolysis in vapor phase, which gives pyrogenic silica, through a wet process, which gives precipitated silica, silica gel, or hydrated silica. The pyrogenic silica is produced essentially in the anhydrous state, while the products of the wet process are obtained as hydrates or contain water absorbed at the surface. Silicon oxide (IV) has a high absorption capacity and can be used in high density liquids. They are used as anti-binders for the same absorption capacity. In addition to this property, silicon oxide (IV) improves the stability of the matrix with which it is interacting, improving its mechanical and rheological properties.

The pigments are used to dye paint, ink, plastic, textiles, cosmetics, food and other products, and are usually in the form of fine powder. This powder is added to a vehicle or matrix, a relatively neutral or colorless material that acts as an adhesive.

A distinction is usually made between a pigment, which is insoluble in the vehicle (forming a suspension), and a dye, which either is a liquid or is soluble in the vehicle (resulting in a solution). A colorant can be a pigment or a dye depending on the vehicle in which it is used. In some cases, a pigment can be manufactured from a dye by precipitating a soluble dye with a metal salt.

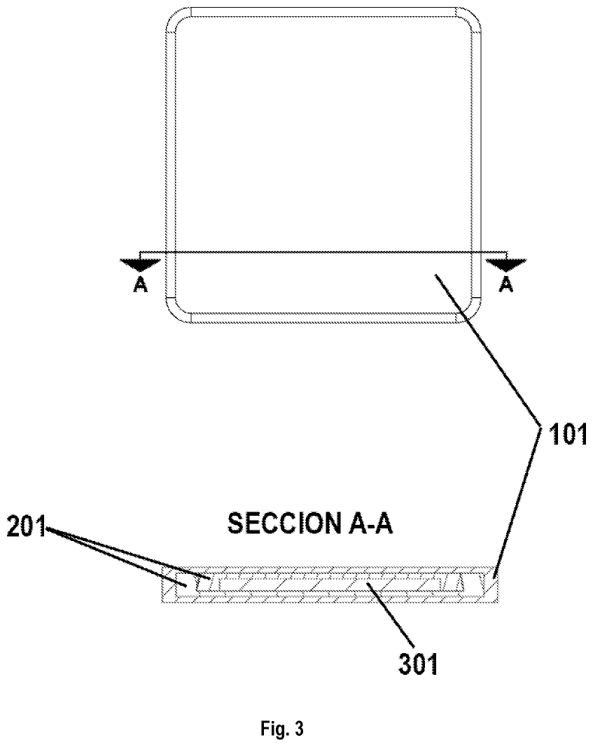

The inner structure (301) will be wrapped by the intermediate structure (201) in its entirety as shown in FIG. 1 and its sectional view as shown in FIG. 3; in turn the intermediate structure (201) is wrapped in the outer structure (101) in its entirety. The purpose of having three structures is that the inner structure (301) contains more fragrance with respect to the outer structure (101) and the intermediate structure (201), which are equal in their composition, the purpose of having the intermediate structure (201) being to generate the support of the inner structure (301) to be adapted to any commercial design of the outer structure (101). By exposing the device in an environment, all the structures will begin to lose fragrance, so that the fragrance that the outer structure (101) loses will be gained by the environment, the fragrance that the intermediate structure (201) loses will be gained by the outer structure (101), and the fragrance that the inner structure (301) loses will be gained by the intermediate structure (201). The foregoing means that the fragrance of the outer structure (101) will be regenerated or refilled; by lengthening its useful life, the outer structure (101) will behave like a re-absorbing membrane that will subsequently release its fragrance to the environment.

The outer structure (101), the intermediate structure (201) and the inner structure (301) of this device may all be made of a variety of polymer matrices within the classification of thermoplastics and elastomers; however, the use of any other material not mentioned in this specification is not limited.

The inner structure (101), the intermediate structure (201) and the outer structure (301) of this device all have material and compounds that make them biodegradable. To achieve the above, it is necessary to use an organic additive called ECO ONE EG35® which is made up of organic compounds, which are incorporated by fusing them to the polymer. This additive can be added within the range of 0.5%-5% by mass with respect to the polymer, which will generate an enveloping film in the polymer molecule, and which facilitates the entry of microorganisms present in biologically active landfills, so that the degradation accelerated by this additive breaks down the polymers and converts them into inert humus (biomass), methane and carbon dioxide.

Preparation Method of the Components of the Device.

The device has three main structures with different specifications, so the method for producing the materials needed for each of them is described below.

Inner Structure (301).

The preparation method of the material that forms the inner structure (301) is the following:
1. The polymer and the fragrance are placed in a tank; this can be done in different ratios such as 4:1; 3:1; 2:1; 1:1; 1:2; 1:3; 1:4, etc. (to mention a few); by mass respectively until saturating the pellet of the polymer that makes up the raw material. This will require from 1 to 72 hours as a deposit time, a longer time not resulting in the pellet absorbing more fragrance. At this stage, a phenomenon called sorption occurs; this refers to the occurrence of adsorption and absorption, these phenomena describing the presence of fragrance on the surface of the pellet and in its inner structure, respectively. The material will be isolated from the environment in a closed device in order to allow it to rest, such resting time lasting from 1 to 72 hours.
2. Once the aforementioned step has been carried out, the material is placed in a flat blade mixer which is operated within a range of 5 to 90 Hz frequency, in order to generate heat which is achieved when two blades move over one another, the movement causing the molecules of the surface regions to move faster, thus raising their temperature by friction. The blade will be gradually heated from 25° C. (room temperature) until reaching a range from 35° C. to 90° C., which achieves an adequate fragrance sorption.
3. Once the minimum temperature mentioned above has been reached, more fragrance will gradually be added until reaching the maximum temperature mentioned above; this can be done in different ratios such as 4:1; 3:1; 2:1; 1:1; 1:2; 1:3; 1:4 (to mention a few); by mass with respect to the mass of the previously wetted pellet, in order to achieve an adequate fragrance sorption, lignocellulosic fiber will be added, from 1% to 50% by mass, previously treated (the fragrance-fiber ratio is proportional, so if more fragrance needs to be added, it will be necessary to put in its equivalent in fiber), the silicon dioxide will be added in a proportion of 0.5% up to 20% by mass (the ratio fragrance-silicon dioxide is proportional, so if more fragrance needs to be added, it will be necessary to put in its equivalent in silicon dioxide); once the mixture is homogenized, it will be placed inside a tank where it will be cooled by natural convection. Once the mixture is at room temperature, it will be placed in a centrifuge, where the speed can range from 5,000-25,000 RPM. Once this step is completed, it will be placed in a closed tank to be used later.

The composites are those materials that are formed by the union of two or more materials to achieve the combination of properties that is not possible to obtain in the original materials.

The materials are composite when they meet the following characteristics:
1. They are formed by two or more physically distinguishable and mechanically separable components.
2. They present several chemically distinct phases, completely insoluble among themselves and separated by an interface.
3. Their mechanical properties are superior to the simple sum of the properties of their components (synergy).
4. Polyphasic materials, such as metal alloys, in which the composition of the present phases is changed through a thermal treatment, do not belong to the composite materials.

Although there is a wide variety of composite materials, the following characteristics can be observed in all of them:
1. Reinforcing agent: it is a phase of discrete character and its geometry is fundamental when defining the mechanical properties of the material. This agent is also called the dispersed phase.
2. Matrix phase or simply matrix: it has a continuous nature and is responsible for the physical and chemical properties. It transmits the efforts to the reinforcing agent. It also protects and gives cohesion to the material.

In the device object of the invention, the continuous phase or matrix is the polymer and the dispersed phase is the fiber, the fragrance and the silicon dioxide. The composite is formed when their bond takes place. Due to the nature of the materials there is a chemical incompatibility between the continuous and dispersed phases; natural fibers are, by nature, lignocellulosic, polar and hydrophilic (mainly due to the presence of —OH groups); silicon dioxide, similarly to fiber, is, by nature, polar and hygroscopic, while many of the polymers are non-polar and hydrophobic; the polarity difference results in poor adhesion between them. This prevents homogeneous dispersion and fiber-matrix adhesion, generating a fiber-matrix interface. This is a free zone between the fiber and the matrix, which is used to achieve an adequate exit of the fragrance towards the intermediate structure (201), subsequently to the outer structure, (101) and finally to the environment. This interface will be improved by silicon dioxide acting as a compatibilizer between fiber and matrix.

Outer Structure (101) and Intermediate Structure (201).

The preparation method of the material that forms the outer structure (101) and the intermediate structure (201) is the following:
1. The polymer is placed in a flat blade mixer, which is operated within a range of 5 to 90 Hz frequency in order to heat the pellet by friction in the same way as for the material of the inner structure (301).
2. The pellet is gradually heated from 25° C. (room temperature) until reaching a range from 35° C. to 90° C., in order to achieve an adequate fragrance sorption.
3. Once this temperature is reached, the fragrance is added; this can be done in different ratios such as 4:1; 3:1; 2:1; 1:1; 1:2; 1:3; 1:4 (to mention a few); by mass, with respect to the mass of the pellet; the silicon dioxide is added in a proportion of 0.5% up to 20% by mass (the fragrance-silicon dioxide ratio is proportional, so if more fragrance needs to be added, it will be necessary to put in its equivalent in silicon dioxide), which will help achieve greater absorption.
4. The pigment is added, once the mixture is homogenized, and the mixture is placed inside a tank, where it is cooled by natural convection. Since the mixture is at room temperature, it is placed in a centrifuge, where the speed can range between 5,000 and 25,000 RPM. Once this step is completed, it will be placed in a closed tank to be used later.

Complete Device.

Once the materials for the two structures have been prepared, the transformation phase of the material is carried out in both components for the inner structure (301) the intermediate structure (201), and the outer structure (101), which process will be carried out by injection.
1. The process is carried out in three steps; in the first step, the inner structure (301) is injected; the size of this structure is not limited since the greater the presence of fragrance needed, the greater the volume of the structure.
2. In the second step, the intermediate structure (201) is injected so as to cover the inner structure (301); in turn the intermediate structure (201) will be made in two parts, and the inner structure (301) will be placed on these 2 parts, the inner structure will be closed and the over-injection will be generated; in this part the material of the intermediate structure (201) will cover the inner structure (301) forming the structure. The size of this structure is limited since the wall will be the carrier of the fragrance released by the inner structure, so that the wall thickness may vary within the range of 0.5 mm to 8.0 mm.
3. Once the device is solidified, it will be stored to avoid exposing it to the environment.
4. In the third step, and having the inner structure (301) inside the intermediate structure (201), the over-injection will be made where now this part will be placed on the mold before closing it, the device being obtained in this manner, which, already solidified, will be stored to avoid its exposure to the environment. The size of this structure is limited since the wall will be the carrier of the fragrance released by the intermediate structure, so that the wall thickness may vary within the range of 0.5 mm to 8.0 mm.
5. When the device needs to be used, it is opened to be exposed to the environment where the desired smell is wanted.

The invention has been described in sufficient detail so that a person with average skill in the art can reproduce and obtain the results that are mentioned in the present invention. However, any person skilled in the art who is competent to implement the present invention may be able to make modifications not described in the present application; therefore, if the subject matter claimed in the following claims is required for the implementation of these modifications in a certain structure or in the manufacturing process thereof, said structure should be understood to be within the scope of the invention.

Having sufficiently described my invention, I consider it novel and therefore claim as my exclusive property what is contained in the following clauses:

1. An encapsulated refill fragrance device for the gradual supply of fragrance to the exposed environment, characterized in that it comprises:
a. One or a plurality of inner structures of any desired geometric shape, which may be formed by any polymer(s) within the category of thermoplastics and/or elastomers to which a compound(s) or compounds that make them biodegradable is(are) added, although this is not limited to the use of another compound not considered in the aforementioned category; which has a continuous phase corresponding to the polymer and a dispersed phase formed by lignocellulosic fibers which have the function of generating the fragrance adsorption, which is a component included in this structure;
b. An outer structure of any desired geometric shape that completely accommodates one or a plurality of intermediate structures and this one totally accommodates a plurality of structures where the outer structure and intermediate structure can be formed by any polymer or various polymers within the category of thermoplastics and/or elastomers to which a compound or compounds that make them biodegradable is added, although this is not limited to the use of any other compound not considered in the aforementioned category and which may or may not contain fragrance as a constituent element of this structure as well as the silicon oxide;
c. The conformation of the inner, intermediate and outer structures are all obtained by an injection process, where a structure or a plurality of inner structures are totally contained internally in the structure and this in turn in an outer structure;
d. In order to give the material used both for the inner and the outer structures the biodegradable characteristic, the use of an organic additive is required which is made up of organic compounds, which is incorporated by fusing it to the polymer; this additive can be added within the range of 0.5%-5% by mass with respect to the polymer, which will generate an enveloping film in the polymer molecule, and which facilitates the entry of microorganisms present in biologically active landfills, so that the degradation accelerated by this additive breaks down the polymers into inert humus or biomass, methane and carbon dioxide.

2. A method for the preparation of the inner, intermediate and outer structures and the complete assembly of the encapsulated refill fragrance device for the gradual supply of fragrance to the exposed environment that comprises:
a. When it comes to the material of the inner structure, the method includes the following steps:
i. The polymer and the fragrance are placed in a tank; this can be done in different ratios such as 4:1; 3:1; 2:1; 1:1; 1:2; 1:3; 1:4, etc. (to mention a few); by mass respectively until saturating the pellet that makes up the raw material, a time of deposit from 1 to 72 hours being allowed for the above step; at this step, a phenomenon called sorption will occur, which refers to the occurrence of adsorption and absorption, these phenomena describing the presence of fragrance on the surface of the pellet and in its inner structure, respectively;
ii. Once the aforementioned step has been carried out, the material is placed in a flat blade mixer; the mixer is operated within a range of 5 to 90 Hz frequency in order to generate heat which is achieved when two blades move one over another; the movement causes the molecules of the surface regions to move faster, thus raising its temperature by friction; the pellet is gradually heated from 25° C. (room temperature) until reaching a range of from 35° C. to 90° C. in order to achieve an adequate fragrance sorption;
iii. Once the aforementioned temperature has been reached, the fragrance will gradually be added, until reaching the maximum temperature mentioned above; this can be done in different ratios such as 4:1; 3:1; 2:1; 1:1; 1:2; 1:3; 1:4 (to mention a few); by mass with respect to the mass of the previously wetted pellet; in order to achieve an adequate fragrance sorption lignocellulosic fiber will be added, from 1% to 50% by mass, previously treated (the fragrance-fiber ratio is proportional, so if more fragrance needs to be added, it will be necessary to put in its equivalent in fiber); the silicon dioxide will be added in a proportion of 0.5% up to 20% by mass (the ratio fragrance-silicon dioxide is proportional, so if more fragrance needs to be added, it will be necessary to put in its equivalent in silicon dioxide); once the mixture is homogenized, it will be placed inside a tank, where it will be cooled by natural convection, since the mixture is at room temperature; it will be placed in a centrifuge, where the speed can range between 5,000 and 25,000 RPM. Once this step is completed, it will be placed in a closed tank to be used later.
b. When it comes to the material of the outer structure and intermediate structure, the method includes the following steps:
i. The polymer is placed in a flat blade mixer, the mixer is operated within a range of 5 to 90 Hz frequency in order to heat the pellet by friction in the same way as for the material of the inner structure;
ii. The pellet is heated gradually from 25° C. (room temperature) until reaching a range that goes from 35° C. to 90° C., which favors the absorption of fragrance;
iii. Once the aforementioned temperature is reached, the fragrance is added; this can be done in different ratios such as 4:1; 3:1; 2:1; 1:1; 1:2; 1:3; 1:4 (to mention a few); by mass, with respect to the mass of the pellet; the silicon dioxide will be added in a proportion of 0.5% up to 20% by mass (the ratio fragrance-silicon dioxide is proportional, so if more fragrance needs to be added, it will be necessary to put in its equivalent in silicon dioxide); this will help achieve greater absorption;
iv. Pigment is added, once the mixture is homogenized, and placed inside a tank, where it is cooled by natural convection, since the mixture is at room temperature; it is then placed in a centrifuge, where the speed can range between 5,000 and 25,000 RPM. Once this step is completed, it will be placed in a closed tank to be used later.
c. When it comes to the complete device, the method includes the following steps once the materials for the three structures mentioned above are manufactured, the transformation phase taking place by an injection process:
i. The process is carried out in three steps; in the first step the internal structure is injected; the size of this structure is not limited, since the greater the presence of fragrance, the greater the volume of the structure will be required,
ii. In the second step, the intermediate structure is injected so that the inner structure is covered; in turn, the intermediate structure will be made in two parts; before starting the injection cycle, the first part will be placed in the mold and the inner structure will be placed on it; this will be closed and the over-injection will be generated; in this part, the material of the intermediate structure will cover the inner structure. The size of this structure is limited since the wall will be the carrier of the fragrance released by the inner structure, so that the wall thickness may vary within a range from 0.5 mm to 8.0 mm;
iii. Once solidified, the device will be stored to avoid exposure to the environment;
iv. In the third step, the inner structure being within the intermediate structure, the over-injection will take place where this part will now be placed on the mold before closing it, the device being obtained in this manner, device which is already solidified and which will be stored to avoid exposure to the environment; the size of this structure is limited since the wall will be the carrier of the fragrance released by the intermediate structure, so that the wall thickness may vary within a range from 0.5 mm to 8.0 mm;
v. When the device needs to be used, it is opened to be exposed to the environment where the desired smell is wanted.
